(19) United States
(12) Patent Application Publication
(10) Pub. No.: US 2008/0221618 A1
(43) Pub. Date: Sep. 11, 2008

(54) CO-EXTRUDED TISSUE GRASPING MONOFILAMENT

(76) Inventors: Gaoyuan Chen, Hillsborough, NJ (US); J. Jenny Yuan, Branchburg, NJ (US); James A. Matrunich, Mountainside, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/684,027

(22) Filed: Mar. 9, 2007

(51) Int. Cl. A61B 17/04; A61L 17/00

(52) U.S. Cl. 606/228; 606/232

(57) ABSTRACT

A co-extruded tissue grasping monofilament and a method for making the same. The monofilament includes a core made of a first material and extending along a length of said monofilament, and a plurality of tissue grasping elements extending outwardly from the core at least along a predetermined portion of the length of the monofilament. The plurality of tissue grasping elements are made of a second, different material having a greater stiffness than the first material. The method for making the monofilament is by co-extrusion.

### FIELD OF THE INVENTION

The present invention relates generally to the field of surgical medical devices, and more particularly to tissue grasping monofilaments comprising at least two co-extruded distinct materials.

### BACKGROUND OF THE INVENTION

Many wound and surgical incisions are closed using surgical sutures or some other surgical closure device. With regard to surgical sutures, various types of barbed sutures have been developed and/or discussed in literature in an effort to help prevent slippage of the suture and/or eliminate at least some knot-tying. With such known barbed sutures, the configuration of the barbs, such as barb geometry (barb cut angle, barb cut depth, barb cut length, barb cut distance, etc.) and/or the spatial arrangement of the barbs, will likely affect the tensile strength and/or holding strength of the suture. There is much prior art focusing on these features, mostly in the context of barbs that are cut into the suture shaft or suture core. In most known monofilament cut, barbed sutures, the tensile strength of a barbed suture is significantly less than a non-barbed suture of equivalent size. This is due to the fact that escarpment of barbs into a monofilament, depending on the barb cut depth, reduces the straight pull tensile strength since the effective suture diameter is decreased. Further, unlike conventional sutures that disproportionately place tension directly at the knots, barbed sutures tend to spread out the tension more evenly along the suture length, including at the location of the barbs. It is therefore critical for the monofilament, at the location of the barbs, to have sufficient tensile strength, and also critical for the barbs themselves to be sufficiently strong to resist breakage or peeling.

Most monofilament barbed sutures are made of relatively soft polymeric materials, thus providing a limit on the stiffness of the barbs. For any given suture size, it is difficult to form barbs large enough and strong enough to catch tissues without bending, slippage or breakage, and without adversely affecting the strength of the suture. The holding strength and tensile strength can be increased by use of a stiffer material for the suture, but any increase in stiffness leads to a decrease in the flexibility of the suture, which is undesirable.

For the foregoing reasons, there is a need for a tissue grasping monofilament having an improved combination of strength and flexibility.

### SUMMARY OF INVENTION

The present invention provides a co-extruded, tissue grasping monofilament having a core made of a first material and extending along a length of the monofilament, and a plurality of tissue grasping elements extending outwardly from the core at least along a predetermined portion of the length of the monofilament. The plurality of tissue grasping elements are made of a second, different material having a greater stiffness than the first material. In one aspect of the invention, the monofilament may be of a size suitable for use as a surgical suture.

According to one embodiment, the second material substantially surrounds the core. In yet another embodiment, the plurality of tissue grasping elements each have a base portion and a distal end portion, with the base portion being embedded within the core. The base portion may further include one or more projections extending laterally outwardly therefrom that assist in mechanically coupling the tissue grasping elements with the core. Further, the cross-section of the plurality of tissue grasping elements may decreases from the proximal end to the distal tip located farthest from the core.

The core may have a substantially uniform cross-section along the length of the monofilament, and may further have a shape that is circular, oval, triangular or polygonal.

In further alternative embodiments, the first material may have an initial modulus of less than or equal to about 400 kpsi, and/or the second material may have an initial modulus of at least about 500 kpsi.

Further, the first material may be a polymeric material such as polyethylene terephthalate, or polymers or copolymers of lactide and glycolide, which may further be 95/5 copolymer of poly(lactide-co-glycolide) or 90/10 copolymer of poly(glycolide-co-lactide). The second material may be a polymeric material such as polypropylene, polydioxanone, or copolymers of poly(glycolide-co-caprolactone), which may further be a 75/25 blocked copolymer of poly(glycolide-co-caprolactone).

According to yet another embodiment the monofilament is formed by co-extrusion of the first and second materials.

Also provided is a method for forming a tissue grasping monofilament including the steps providing a first material having a first stiffness in its solid state, providing a second material having a second, different stiffness in its solid state that is greater than that of the first material, melting the first material and extruding the melted first material through a first die having a predetermined shape to form a first melt stream having substantially the predetermined shape, melting the second material and introducing the melted second material into a merging chamber having the first melt stream passing therethrough such that the second material substantially surrounds said first melt stream, extruding the first melt stream surrounded by the melted second material together through a second die having a predetermined shape with an outer periphery greater than an outer periphery of the first die and with at least one ridge extending outwardly beyond the outer periphery of the first die, and cooling said first and second materials to form a solid monofilament. The method may further include the step(s) of drawing the cooled monofilament to form an oriented monofilament, and/or, following cooling, forming tissue grasping elements along a predetermined length of the second material by removing material from the at least one ridge formed of the second material.

In one embodiment, the predetermined shape of the first die is substantially oval or circular.

In yet another embodiment, the first material has an initial modulus of less than or equal to about 400 kpsi, and the second material has an initial stiffness of at least about 500 kpsi.

The first material may further be a polymeric material such as polyethylene terephthalate or polymers or copolymers of lactide and glycolide, and the second material may further be a polymeric material such as polypropylene, poydioxanone, or copolymers of poly(glycolide-co-caprolactone).

A further method is provided including the steps of providing a first material having a first stiffness in its solid state, providing a second different material having a second stiffness in its solid state that is greater than that of the first material, melting the first and second materials, and co-extruding the first and second materials to form a monofilament wherein the first material forms a core of the monofilament and the second material forms one or more ridges extending outwardly beyond an outer periphery of the core.

According to this method the second material of the co-extruded monofilament may further substantially surround the core.

In yet another embodiment, a base portion of each of the plurality of ridges may further be embedded within the core and a distal end portion of each of the plurality of ridges extend outwardly beyond the outer periphery of the core. The base portion each of the plurality of ridges may further include one or more projections extending laterally outwardly therefrom.

In yet another embodiment, the method further includes forming a plurality of tissue grasping elements in the one or more ridges by removing material therefrom at predetermined locations.

In additional alternative embodiments, the core of the monofilament may have a substantially oval or circular shape, and/or the first material may have an initial modulus of less than or equal to about 400 kpsi, and the second material may have an initial stiffness of at least about 500 kpsi.

The first material may further be a polymeric material such as polyethylene terephthalate or polymers or copolymers of lactide and glycolide, and the second material may be a polymeric material such as polypropylene, polydioxanone or copolymers or poly(glycolide-co-caprolactone).

### BRIEF DESCRIPTION OF THE DRAWINGS

The invention will now be described in more detail with reference to the accompanying drawings, in which:

FIG. 1a is a schematic illustration of an exemplary co-extrusion process that can be used to form monofilaments according to the present invention;

FIG. 1b is a cross-section of one embodiment of a monofilament of the present invention;

FIGS. 1c and 1d are perspective views of the monofilament of FIG. 1b before and after tissue grasping elements are formed;

FIGS. 1e-1f are cross-sectional views illustrating alternate embodiments of the monofilament of the present invention;

FIG. 2 is a schematic illustration of an exemplary drawing process that can be used to form monofilaments according to the present invention;

FIG. 3a-3d are cross-sectional views of various embodiments of a monofilament according to the present invention wherein the tissue grasping elements are at least partially embedded within the core;

FIG. 4 illustrates a cross-section of an embodiment of a monofilament according to the present invention wherein the tissue grasping elements are formed on and adhere to the outer periphery of the core; and

FIG. 5 illustrates an exemplary cut that can be used in forming tissue grasping elements on a monofilament according to the present invention.

### DETAILED DESCRIPTION

By way of background and as those skilled in the art recognize, “extrusion” typically refers to a polymer processing technique in which a polymer is melted and pressurized in an extruder, and fed through a die in a continuous stream. For purposes of the present application, the term “co-extrusion” refers to a process where two or more different materials, such as polymers, are melted in separate extruders with both melt streams fed through a co-extrusion die wherein they are joined to form a single molten strand. Further, the term “stiffness” as used herein refers the load required to deform a material, which is measured by the slope of the stress-strain curve. The initial slope of the stress-strain curve (typically from 0.5% -1.5% strain range) is also termed as Young's Modulus or the initial modulus, which is the measure of stiffness used herein.

Tissue grasping monofilament medical devices according to the present invention comprise at least two different components that are co-extruded. The term “different” as used herein is intended to cover both distinctly different materials having fundamentally different chemical formulas and structures, or materials having similar chemical formulas and structures, but different molecular weights and thus potentially different physical properties. The first component forms a core or shaft and the second component forms the tissue grasping elements, or one or more “ridges” extending substantially lengthwise along a predetermined length of the filament, and out of which the tissue grasping elements are formed by cutting or otherwise removing portions of the ridge. The cross-section of the core may be any shape including, but not limited to, round, oval, triangle, square or rectangular. The cross-section of the ridge and ultimately the tissue grasping elements can also be of substantially any shape suitable to increase the holding strength of the monofilament. Particularly suitable configurations of the ridge are triangular or various other shapes that have a wider base than distal end. The core and the ridges may be coupled simply by adherence of the two dissimilar materials together during the co-extrusion process, or may be physically reinforced by complementary interlocking shapes as will be described further below. By co-extruding two different materials and optimally selecting the materials as described herein, a tissue grasping monofilament can be achieved having both improved strength of the tissue grasping elements, and an improved combination of tensile strength and flexibility.

The two materials may be made from various suitable biocompatible materials, such as absorbable or non-absorbable polymers. The two materials may have different properties, such as modulus, strength, in vivo degradation rates, so that the desired properties for overall performance of the tissue grasping monofilament device and the capability of the tissue grasping elements to engage and maintain wound edges together can be tailored. Preferably, the first component is a relatively soft material having an initial modulus of no greater than about 400 kpsi and the second component is a stiffer material having an initial modulus of at least about 500 kpsi. Preferable materials for the second component include, but are not limited to, polyethylene terephthalate, polymers or copolymers of lactide and glycolide, and more preferably 95/5 copolymer of poly (lactide-co-glycolide), 90/10 copolymer of poly (glycolide-co-lactide), and materials for the first component include, but are not limited to, polypropylene, polydioxanone, copolymers of poly (glycolide-co-caprolactone).

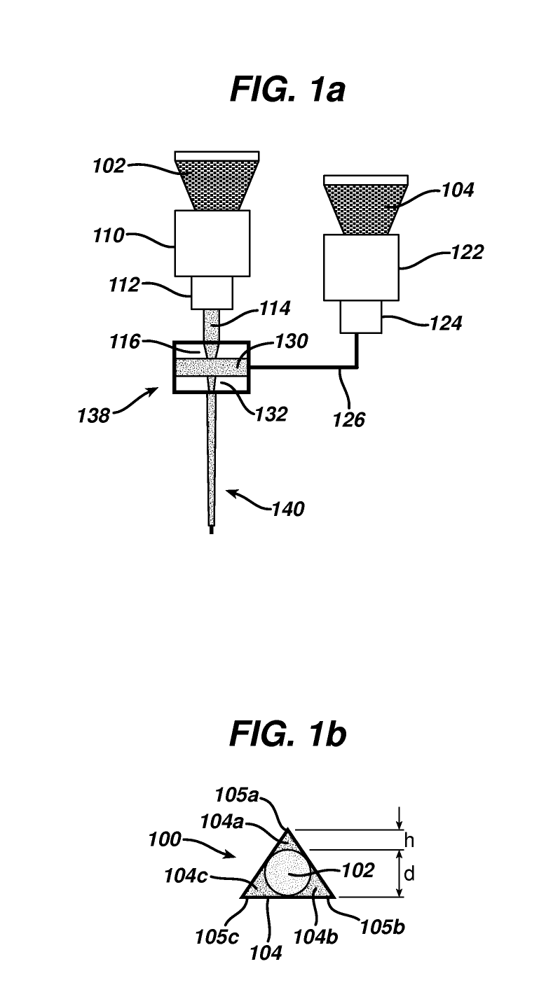

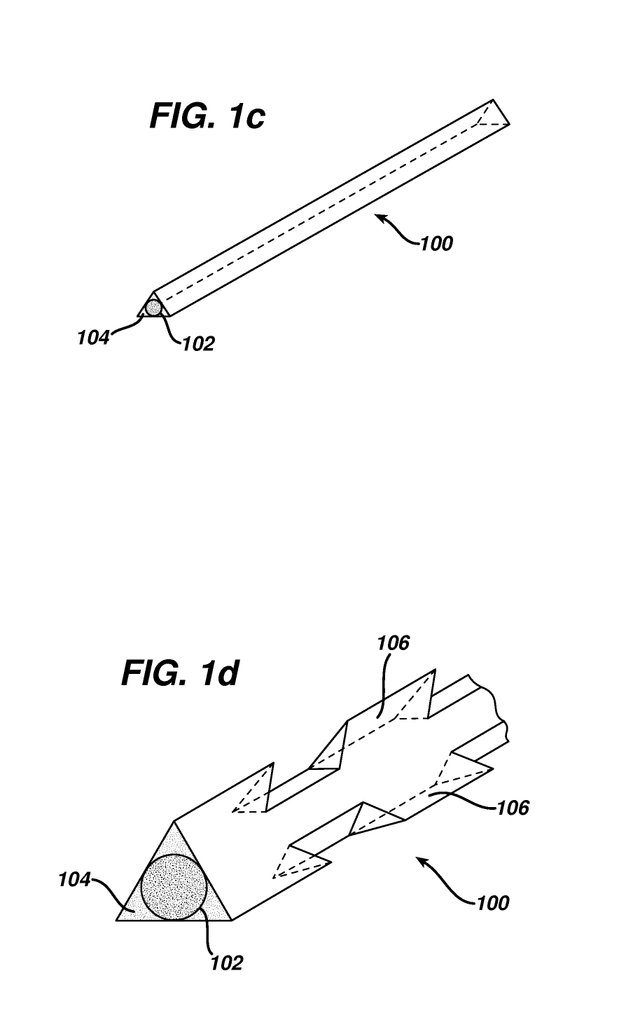

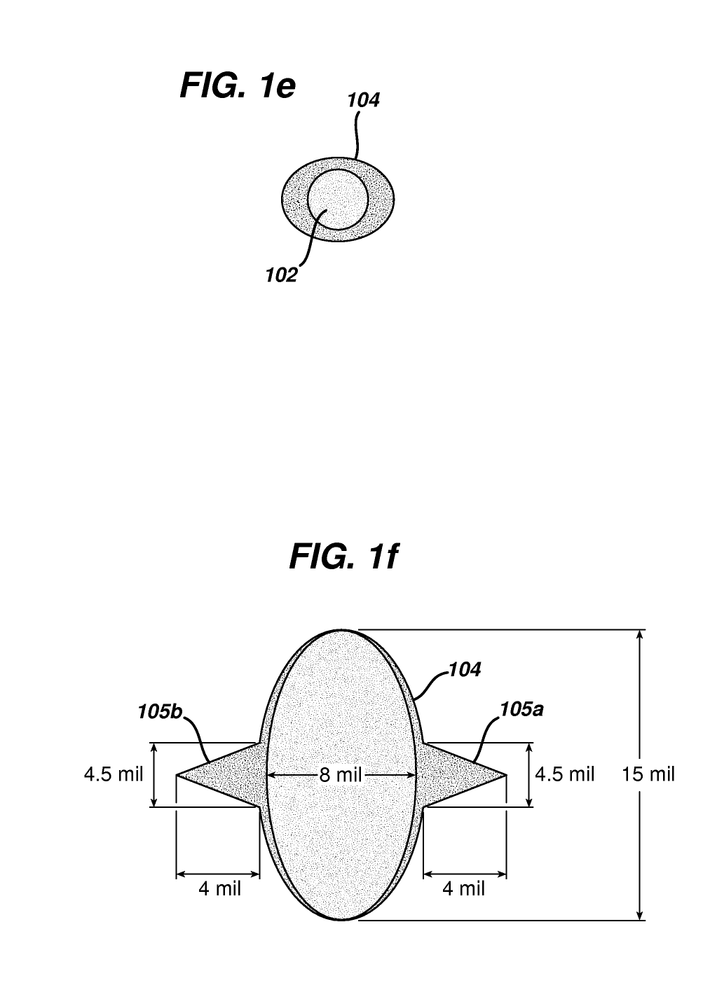

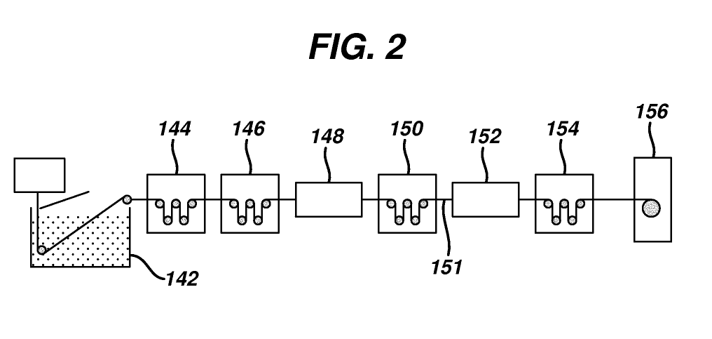

FIGS. 1b-d illustrate one exemplary embodiment of a co-extruded tissue grasping monofilament 100 according to the present invention. In this embodiment, the first component 102 is made of polydioxanone (PDS), and the second component 104 is made of polylactide (PLA) and polyglycolide (PGA) or 95/5 PLA/PGA copolymers (a stiffer material with a higher initial modulus). The second component has a substantially triangular overall outer perimeter forming first, second and third 104a, 104b, 104c ridges extending outwardly from the core 102. Tissue grasping elements 106 subsequently cut into the ridges are shown in FIG. 1d. With a co-extruded monofilament wherein the second material has a greater stiffness, the holding strength of the tissue grasping elements is greater due to the greater stiffness. In the illustrated embodiment, the core is substantially circular in cross-section and has an outer diameter d of approximately 2-30 preferably 5-25 mil. Further, each ridge and resulting tissue grasping elements projects outwardly from the core to a distal tip 105a, 105b, 105c a distance h of approximately 3-50 mil, preferably 8-35 mil.

Referring now to FIG. 1a, one exemplary process for making a co-extruded monofilament of the type shown in FIGS. 1b-d will now be described in detail. The first component, which as indicated can be PDS, is melted in a first extruder 110, metered and pressurized through a gear pump 112. The pressurized polymer melt stream 114 (which is inside a heated metal block or a transfer tube, not shown) passes through an upper die 116 of a shape suitable to form the desired cross-section of the core, in this case circular. The second component (i.e., PLA) 104 is melted in a second extruder 122, metered, and pressurized through the gear pump 124. The second pressurized polymer melt stream 126 (inside a heated transfer tube, not shown) enters a merging chamber 130 in the co-extrusion die block 138 between the upper die 116 and a lower die 132. More specifically, as used herein the term “merging chamber” refers to the portion of the co-extrusion die block 138 where the melt streams of the first and second components merge before being extruded together through the bottom or lower die 132. At a given temperature, the lower modulus material has a lower viscosity, which aids in its ability to flow around the core component before entering the lower die. The merged stream 134 of the two components passes through the lower die 132 of a predetermined shape (in this case triangular) to form the desired overall cross-section of the co-extruded monofilament 140.

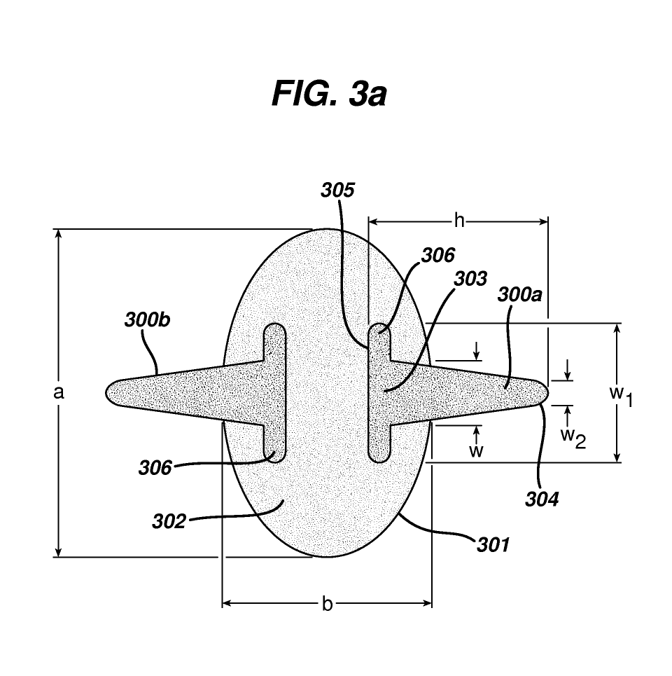

The co-extruded molten monofilament strand 140 exiting the co-extrusion die block 138 is quenched and solidified in a liquid bath 142 as illustrated in FIG. 2, to quickly preserve the shape of the extrudate. The solidified dual-component monofilament strand is then passed through a first set of godet rolls 144 at a constant speed and then drawn or stretched preferably to 2-10 times its original length with the second set of feeding or godet rolls 146 running at a faster speed. As is well known, drawing or stretching (as opposed to injection molding techniques) improves strength by orienting molecules along the axis of the fiber. The drawn strand may be drawn for the second time with the third set of rolls 150 to reach the maximum stable draw ratio to optimize the tensile properties. During the drawing process, the monofilament can be heated with one or several of the feeding rolls and/or through a hot oven 148. The fully drawn monofilament 151 may then be relaxed by passing through a heated relaxation oven 152 and onto another set of rolls 154 running at a slightly slower speed before taking up with winding device 156.

The co-extrusion process described above, in combination with natural adherence between the two materials, mechanically couples the two components to result in a suitable co-extruded monofilament. The core of the first, less stiff material allows for good overall flexibility of the monofilament, while the second, stiffer material into which the tissue grasping elements are formed allows for stronger tissue grasping elements leading to better holding strength for the monofilament. Finally, because the suture core 102 remains intact, tensile strength is not adversely affected.

Although a substantially triangular overall cross-section is illustrated in FIG. 1b, it is to be understood that any suitable cross-section can be used and achieved with co-extrusion, such as, but not limited to, circular or oval as shown in FIGS. 1e and 1f, or any suitable polygonal cross-section. The cross-section of the core may be varied as well.

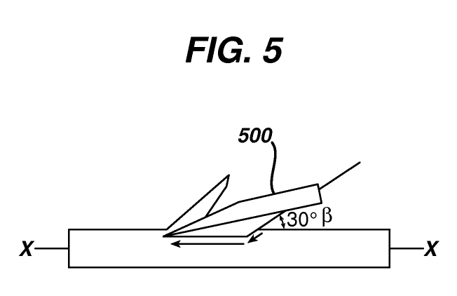

As previously indicated, the tissue grasping elements can be formed in the ridges in any suitable configuration and by any suitable manner known to those skilled in the art, such as cutting by knife, laser or other device, stamping, punching, press forming or the like. For example, in one embodiment the tissue grasping elements are formed by cutting with a suitable cutting blade or knife. The desired number of acute, angular cuts are made directly into the ridges of the co-extruded monofilament. FIG. 5 illustrates an exemplary cut, where the cutting blade 500 first cuts into the ridge at an angle β of approximately 30 degrees relative to the longitudinal axis x-x of the monofilament, to a depth approximately equal to or preferably less than the height of the ridges, and subsequently further cuts into the monofilament for a distance of approximately 50%˜100% of the height of the ridges at an angle of approximately 0 degrees. To facilitate this cutting, the monofilament is typically placed and held on a cutting vice or the like. A template may also be used to help guide the cutting blade. As the ridges protrude from the core, an alternate means for cutting the tissue grasping elements is to slice across the ridges from one side to the other, thus making it a one motion movement cutting and increasing efficiency. The blade will take the shape of the tissue grasping element configuration with the cutting blade on the side instead of in the front. Also, since the tissue grasping element configuration is pre-determined by the shape of the blades, the changes can easily be made to the machine if changes are desired. As indicated, material can be removed from the ridges by other suitable means such as laser cutting or stamping.

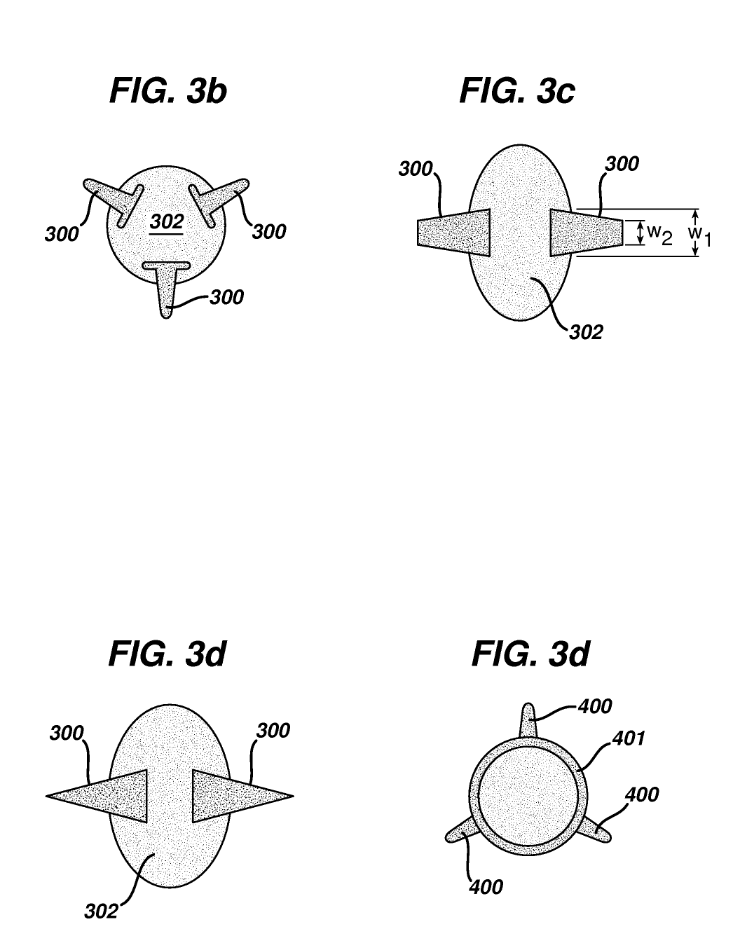

Referring now to FIGS. 3a-d, in alternate embodiments according to the present invention, the second component from which the tissue grasping elements are formed does not surround the core, but rather is mechanically coupled with the core and projects outwardly therefrom. For example, as shown in FIG. 3a, first and second ridges 300a, 300b (within which the tissue grasping elements are subsequently formed) extend outwardly from the core 302, but a base portion 303 at a proximal end thereof is embedded within the core. Preferably, each is configured to provide additional mechanical resistance against pulling the tissue grasping elements out of the core. In FIGS. 3a-c, the ridges include the base portion 303 that is larger in width w₁ and cross-section than the width w₂ and cross-section of the distal tip portion 304. The base portion may include additional extensions or projections 306 that extend laterally outward and assist in mechanically locking the projection to the core. As stated, embedding the ridges into the core provides additional security through “mechanical locking” between the ridge material and core material. The two ridges preferably are placed along the short axis of the oval core if the core is oblong, so as to minimally affect overall stiffness of the monofilament. Further, in the illustrated exemplary embodiment, the overall dimension of the core is approximately 4-40 mil, preferably 15 mil (dimension a) by approximately 2-20 mil, preferably 8 mm (dimension b), and dimensions w, w₁ and w₂ are approximately 1-10 mil, preferably 4 mil, 2-20 and preferably 8 mil, and 0.4-4, preferably 1.5 mil respectively.

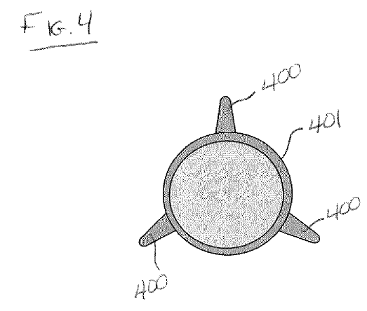

As further shown in FIGS. 3b-3d, the number of ridges 300 and/or their configurations can vary to best suit the desired product features in a given surgical application. In addition, the core can take circular and non-circular cross-sections to accommodate the number of ridges, mechanical properties of the filaments, and the extrusion process. Further, similar type ridges 400 extending from the outer periphery of the core can be connected by a relatively thin membrane or covering 401 of the same material that surrounds or substantially surrounds the core as shown in FIG. 4.

The following are detailed representative examples of co-extruded, tissue grasping monofilaments of the present invention which are exemplary only, as the present invention is not intended to be limited other than by the appended claims.

### EXAMPLE 1

A nonabsorbable tissue grasping monofilament substantially of the configuration shown in FIG. 1b was formed using the coextrusion process shown and described above in connections with FIGS. 1a and 2. Polypropylene (PP) was used as the first component with has an initial modulus of 236 kpsi in the oriented fiber of the homopolymer. Polyethylene terephthalate (PET) with an initial modulus of 2044 kpsi, was used for the second component.

As shown in FIG. 1a, the first component, PP, was melted in a first extruder 110, where the extruder barrel had three temperature zones maintained, respectively, at 180, 195 and 210° C. The melted polymer stream was metered and pressurized through a gear pump 112 and the pressurized polymer melt stream 114 passed through a circular upper die 116 to form a circular core. The second component (PET) was melted in a second extruder 122 maintained at a constant temperature of 285° C. in all three zones. The melt flow was then metered, and pressurized through the gear pump 124. The second pressurized polymer PET melt stream 126 entered a merging chamber 130 in the co-extrusion die block 138 between the upper die 116 and a lower die. The merged stream 134 of the two components passes through the lower die 132 of a triangular shape to form a triangular overall cross-section of the co-extruded monofilament 140.

The co-extruded molten PP/PET monofilament strand 140 exiting the co-extrusion die block 138 was quenched and solidified in a liquid bath 142 as illustrated in FIG. 2. The solidified PP/PET dual-component monofilament strand was then passed through a first set of godet rolls 144, the last two of which were heated at a temperature of 122° C. The feeding speed was 122 feet per minute (fpm). The co-extruded monofilament was passed to and drawn with the second set of godet rolls 146 running at a speed of 50.5 (no heating was applied). The partially stretched strand was drawn again with the third set of rolls 150 running at 57 fpm. The total draw ratio was 6.0. The hot oven 148 was six feet long and was heated at 135° C. The fully drawn monofilament 151 was relaxed by passing through a six-foot oven 152 maintained at 135° C. and onto another set of rolls 154 running at a speed of 57 fpm before taking up with winding device 156.

Tissue grasping elements were subsequently formed by cutting along the three ridges of essentially PET to form a tissue grasping monofilament having a less stiff, more pliable core while having stiffer, more rigid tissue grasping elements.

### EXAMPLE 2

A substantially identical configuration and process as Example 1, the exception that the second component was a 90/10 PGA/PLA random copolymer with an initial modulus of 914 kpsi and an absorption time of 50-70 days. The first component was a 75/25 PGA/PCL block copolymer with an initial modulus of 106 kpsi and an absorption time of 91-119 days. The two polymer components were found to have been adequately connected via adhesion at their interfaces. Tissue grasping elements were formed as described above.

Although illustrative embodiments of the present invention have been described herein with reference to the accompanying drawings, it is to be understood that the invention is not limited to those precise embodiments and that various other changes and modifications may be effected herein by one skilled in the art without departing from the scope or spirit of the invention.

1. A co-extruded, tissue grasping monofilament, comprising:
a core comprised of a first material and extending along a length of said monofilament; and
a plurality of tissue grasping elements extending outwardly from said core at least along a predetermined portion of the length of the monofilament, the plurality of tissue grasping elements being comprised of a second, different material having a greater stiffness than the first material.

2. The monofilament according to claim 1, wherein the monofilament is of a size suitable for use as a surgical suture.

3. The monofilament according to claim 1, wherein the second material substantially surrounds the core.

4. The monofilament according to claim 1, wherein the plurality of tissue grasping elements each have a base portion and a distal end portion, and wherein the base portion is embedded within the core.

5. The monofilament according to claim 4, wherein the base portion has one or more projections extending laterally outwardly therefrom that assist in mechanically coupling the tissue grasping elements with the core.

6. The monofilament according to claim 1, wherein a cross-section of the plurality of tissue grasping elements decreases from a proximal end thereof to a distal tip thereof located farthest from said core.

7. (canceled)

8. The monofilament according to claim 7, wherein the cross-section of the core is a shape selected from the group consisting of circular, oval, triangular and polygonal.

9. The monofilament according to claim 1, wherein the first material has an initial modulus of less than or equal to about 400 kpsi.

10. The monofilament according to claim 9, wherein the second material has an initial modulus of at least about 500 kpsi.

11. The monofilament according to claim 10, wherein the first material is a polymeric material selected from the group consisting of polyethylene terephthalate, and polymers or copolymers of lactide and glycolide.

12. The monofilament according to claim 11, wherein the copolymers of lactide and glycolide is a polymeric material selected from the group consisting of 95/5 copolymer of poly(lactide-co-glycolide) and 90/10 copolymer of poly(glycolide-co-lactide).

13. The monofilament according to claim 11, wherein the second material is a polymeric material selected from the group consisting of polypropylene, polydioxanone, and copolymers of poly(glycolide-co-caprolactone).

14. The monofilament according to claim 13, wherein the second material is a 75/25 blocked copolymer of poly(glycolide-co-caprolactone).

15. (canceled)

16. A method for forming a tissue grasping monofilament comprising the steps of:
providing a first material having a first stiffness in its solid state;
providing a second material having a second, different stiffness in its solid state that is greater than that of the first material;
melting the first material and extruding the melted first material through a first die having a predetermined shape to form a first melt stream having substantially the predetermined shape;
melting the second material and introducing the melted second material into a merging chamber having the first melt stream passing therethrough such that the second material substantially surrounds said first melt stream;
extruding the first melt stream surrounded by the melted second material together through a second die having a predetermined shape with an outer periphery greater than an outer periphery of the first die and with at least one ridge extending outwardly beyond the outer periphery of the first die; and
cooling said first and second materials to form a solid monofilament.

17. The method according to claim 16, further comprising drawing the cooled monofilament to form an oriented monofilament, and following cooling, forming tissue grasping elements along a predetermined length of the second material by removing material from the at least one ridge formed of the second material.

18. (canceled)

19. (canceled)

20. The method according to claim 16, wherein the first material has an initial modulus of less than or equal to about 400 kpsi, and the second material has an initial stiffness of at least about 500 kpsi.

21. The method according to claim 20, wherein the first material is a polymeric material selected from the group consisting of polyethylene terephthalate and polymers or copolymers of lactide and glycolide, and the second material is a polymeric material selected from the group consisting of polypropylene, poydioxanone, and copolymers of poly(glycolide-co-caprolactone).

22. A method for forming a monofilament comprising the steps of:
providing a first material having a first stiffness in its solid state;
providing a second different material having a second stiffness in its solid state that is greater than that of the first material;
melting the first and second materials;
co-extruding the first and second materials to form a monofilament wherein the first material forms a core of the monofilament and the second material forms one or more ridges extending outwardly beyond an outer periphery of the core.

23. The method according to claim 22, wherein the second material of the co-extruded monofilament substantially surrounds the core.

24. The method according to claim 22, wherein a base portion of each of the plurality of ridges is embedded within the core and a distal end portion of each of the plurality of ridges extends outwardly beyond the outer periphery of the core.

25. The method according to claim 24, wherein the base portion each of the plurality of ridges further includes one or more projections extending laterally outwardly therefrom.

26. The method according to claim 22, further comprising forming a plurality of tissue grasping elements in the one or more ridges by removing material therefrom at predetermined locations.

27. (canceled)

28. The method according to claim 22, wherein the first material has an initial modulus of less than or equal to about 400 kpsi, and the second material has an initial stiffness of at least about 500 kpsi.

29. The method according to claim 28, wherein the first material is a polymeric material selected from the group consisting of polyethylene terephthalate and polymers or copolymers of lactide and glycolide, and the second material is a polymeric material selected from the group consisting of polypropylene, polydioxanone and copolymers or poly(glycolide-co-caprolactone).

30. A co-extruded monofilament, comprising:
a core comprised of a first material extending along a length of said monofilament; and
an outer portion comprised of a second material that is different than the first material, the outer portion surrounding an outer periphery of the core and having a cross-section greater than a cross-section of the core,
wherein the cross-section of the outer portion is substantially circular and the cross-section of the core is substantially triangular.
